(19) United States
(12) Patent Application Publication
(10) Pub. No.: US 2023/0032465 A1
(43) Pub. Date: Feb. 2, 2023

(54) ANTIBODY-DRUG CONJUGATES SPECIFIC FOR CD276 AND USES THEREOF

(71) Applicant: The U.S.A., as Represented by the Secretary, Department of Health and Human Services, Bethesda, MD (US)

(72) Inventors: Brad St. Croix, Frederick, MD (US); Yang Feng, Frederick, MD (US); Steven Seaman, Martinsburg, WV (US)

(73) Assignee: The U.S.A., as represented by the Secretary, Department of Health and Human Services, Bethesda, MD (US)

(21) Appl. No.: 17/783,171

(22) PCT Filed: Dec. 8, 2020
(86) PCT No.: PCT/US2020/063732

§ 371 Date: Jun. 7, 2022

Related U.S. Application Data
(60) Provisional application No. 62/947,135, filed on Dec. 12, 2019.

(51) Int. Cl. C07K 16/28; A61K 47/68; A61P 35/00

(52) U.S. Cl. CPC C07K 16/2827; A61K 47/6803; A61K 47/6849; A61K 47/6889; A61P 35/00

(57) ABSTRACT

An improved antibody-drug conjugate (ADC) targeting CD276-positive tumors is described. The ADC includes a CD276-specific IgG1 antibody having a heavy chain modified to prevent interaction of its Fc domain with endogenous Fc receptors and to introduce a cysteine for site-specific conjugation of the drug. The CD276-specific ADC is capable of potently eradicating CD276-positive tumors in several animal models.

### CROSS REFERENCE TO RELATED APPLICATIONS

This application claims the benefit of U.S. Provisional Application No. 62/947,135, filed Dec. 12, 2019, which is herein incorporated by reference in its entirety.

### ACKNOWLEDGMENT OF GOVERNMENT SUPPORT

This invention was made with government support under project number ZIA BC 010578 awarded by the National Institutes of Health. The government has certain rights in the invention.

### FIELD

This disclosure concerns an improved antibody-drug conjugate (ADC) that targets CD276 (B7-H3) and its use in the treatment of CD276-expressing tumors.

### BACKGROUND

CD276, also known as B7-H3, is a type I transmembrane protein expressed on the surface of many different cell types, including cells of the immune system, liver, heart, prostate, spleen and thymus (Picarda et al., Clin Cancer Res 22(14): 3425-3431, 2016). The CD276 protein is also overexpressed in several human malignancies, including hepatocellular carcinoma, melanoma, leukemia, breast cancer, prostate cancer, colorectal cancer, osteosarcoma, endometrial cancer, ovarian cancer, oral squamous cell carcinoma, non-small cell lung cancer, bladder cancer and pancreatic cancer (Picarda et al.). Expression of CD276 is positively correlated with cancer severity and patient outcome for many types of cancer. Due to its expression pattern and functional activity, CD276 has become a target of interest for cancer immunotherapy.

Antibody-drug conjugates (ADCs) are one type of molecule currently being investigated as therapeutic agents for the treatment of cancer. An ADC is comprised of an antibody (or antigen-binding fragment) conjugated to a cytotoxic compound. The most effective ADCs specifically target tumor cells or tumor-associated stromal cells, include a drug that is highly toxic for tumor cells with minimal activity against normal cells, are highly stable in the circulation, and can release the drug upon internalization into target tumor cells. Radiolabeled monoclonal antibodies that specifically bind CD276 are currently under clinical investigation for the treatment of cancer (NCT01099644, NCT01502917 and NCT00089245; Picarda et al.; and Kramer et al., J Neurooncol 97: 409-418, 2010).

### SUMMARY

Disclosed herein are antibody-drug conjugates (ADCs) that specifically target CD276-expressing tumor cells. Use of the ADCs for treating CD276-positive cancer is also disclosed.

Provided herein are ADCs that include a drug conjugated to a monoclonal antibody that specifically binds CD276. In some embodiments, the ADC includes a variable heavy (VH) domain, a variable light (VL) domain and an IgG1 Fc region, wherein the VH domain includes the complementarity determining region 1 (CDR1), CDR2 and CDR3 sequences of the m276 antibody VH domain (SEQ ID NO: 2), the VL domain includes the CDR1, CDR2 and CDR3 sequences of the m276 antibody VL domain (SEQ ID NO: 6), and the Fc region comprises S239C, L234A, L235A and P329G mutations; and the drug is conjugated to the cysteine at residue 239 of the Fc domain by site-directed conjugation. In some examples, the drug includes pyrrolobenzodiazepine (PBD), such as a PBD dimer. In some examples, the drug is conjugated to the monoclonal antibody via a linker that includes a maleimide group, polyethylene glycol (PEG) and a valine-alanine dipeptide.

Compositions that include an ADC disclosed herein and a pharmaceutically acceptable carrier are also provided.

Further provided are methods of treating a CD276-positive cancer in a subject, and methods of inhibiting tumor growth or metastasis of a CD276-positive cancer in a subject. In some embodiments, the methods include administering a therapeutically effective amount of an ADC or composition disclosed herein.

The foregoing and other objects and features of the disclosure will become more apparent from the following detailed description, which proceeds with reference to the accompanying figures.

### BRIEF DESCRIPTION OF THE DRAWINGS

FIG. 1: m276-PBD-SL elicits potent antitumor activity against human neuroblastoma xenograft tumors grown subcutaneously in mice. Treatment with m276-PBD-SL (right panel) was initiated when tumors reached an average size of approximately 1200 mm³. Animals were administered 0.5 mg/kg m276-PBD-SL once per week starting on the day indicated (arrow). Untreated animals were used as controls (left panel). Each line represents the growth of an individual tumor. N=6/group (untreated control) or 4/group (m276-PBD-SL treated).

FIG. 2: m276-PBD-SL elicits potent antitumor activity against a second human neuroblastoma xenograft tumor model grown subcutaneously in mice. Treatment with vehicle (left panel) or m276-PBD-SL (right panel) was initiated when tumors reached an average size of approximately 1000 mm³. Animals were administered vehicle or 0.5 mg/kg m276-PBD-SL once per week starting on the day indicated (arrows). Each line represents the growth of an individual tumor. N=8/group (vehicle) or 7/group (m276-PBD-SL treated).

FIG. 3: m276-PBD-SL elicits potent antitumor activity against a human breast xenograft tumor model grown orthotopically in mice. Treatment with vehicle or m276-PBD-SL was initiated when tumors reached an average size of approximately 1000 mm³. Animals were administered vehicle (left panel), 0.1 mg/kg m276-PBD-SL (middle panel) or 0.5 mg/kg m276-PBD-SL (right panel) once per week starting on the day indicated (arrows). Each line represents the growth of an individual tumor. N=10/group (vehicle) or 11/group (0.1 and 0.5 mg/kg m276-PBD-SL treated).

FIG. 4: Schematic comparison of the linkers used for the m276-PBD and m276-PBD-SL ADCs.

FIG. 5: Orthotopic Py230 breast tumors initially regress and then relapse after treatment with m276-PBD glycoconjugate. Mice bearing Py230 tumors were administered vehicle (left) or 1 mg/kg m276-PBD twice per week for four weeks. Treatment was initiated when the average tumor volume reached 140 mm³. Each line represents the tumor growth from an individual mouse. All tumors relapsed in mice treated with m276-PBD.

FIG. 6: Large orthotopic MDA-MB-231 breast cancer tumors show complete response after treatment with m276-PBD-SL. Mice bearing MDA-MB-231 tumors were administered vehicle (left), 0.1 mg/kg m276-PBD-SL (middle) or 0.5 mg/kg m276-PBD-SL (right) once per week for five weeks. Treatment was initiated when the average tumor volume reached 1000 mm³. Each line represents the tumor growth from an individual mouse. The data show that relapse occurred in some of the mice treated with the lower (0.1 mg/kg) dose, but complete responses were observed in all mice treated with the higher (0.5 mg/kg) dose of m276-PBD-SL.

FIG. 7: Large orthotopic SUM159 breast cancer tumors show complete response after treatment with m276-PBD-SL. Mice bearing SUM159 tumors were administered vehicle (left) or 0.5 mg/kg m276-PBD-SL (right) once per week for four weeks. Treatment was initiated when the average tumor volume reached 1000 mm³. Each line represents the tumor growth from an individual mouse. The results demonstrate that treatment with m276-PBD-SL led to complete regression of SUM159 tumors.

### SEQUENCE LISTING

The amino acid sequences listed in the accompanying sequence listing are shown using standard three letter code for amino acids, as defined in 37 C.F.R. 1.822. The Sequence Listing is submitted as an ASCII text file, created on Nov. 17, 2020, 15.3 KB, which is incorporated by reference herein. In the accompanying sequence listing:

SEQ ID NO: 1 is the amino acid sequence of a signal peptide.
MEWSWVFLFFLSVTTGVHS
SEQ ID NO: 2 is the amino acid sequence of the m276 variable heavy (VH)
domain. CDR sequences are indicated by bold underline.
QVQLQQSGAEVKKPGSSVKVSCKASGGTFSSYAISWVRQAPGQGLEWMGGIIPILGIANY
AQKFQGRVTITADESTSTAYMELSSLRSEDTAVYYCARGGSGSYHMDVWGKGTTVTVSS
SEQ ID NO: 3 is the amino acid sequence of the modified m276 constant region.
Modified residues are indicated by bold underline.
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPEAAGGPC
VFLFPPKPKDTLMISRTPEVTCVVVDVSHEDPEVKFNWYVDGVEVHNAKTKPREEQYNST
YRVVSVLTVLHQDWLNGKEYKCKVSNKALGAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 4 is the amino acid sequence of the modified m276 heavy chain.
Modified residues are indicated by bold underline.
QVQLQQSGAEVKKPGSSVKVSCKASGGTFSSYAISWVRQAPGQGLEWMGGIIPILGIANYA
QKFQGRVTITADESTSTAYMELSSLRSEDTAVYYCARGGSGSYHMDVWGKGTTVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPCVF
LFPPKPKDTLMISRTPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTLPPSRDELTKN
QVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 5 is the amino acid sequence of the modified m276 heavy chain
with an N-terminal signal peptide (underlined).
MEWSWVFLFFLSVTTGVHSQVQLQQSGAEVKKPGSSVKVSCKASGGTFSSYAISWVRQAP
GQGLEWMGGIIPILGIANYAQKFQGRVTITADESTSTAYMELSSLRSEDTAVYYCARGGSG
SYHMDVWGKGTTVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEPKSCD
KTHTCPPCPAPEAAGGPCVFLFPPKPKDTLMISRTPEVTCVVVDVSHEDPEVKFNWYVDG
VEVHNAKTKPREEQYNSTYRVVSVLTVLHQDWLNGKEYKCKVSNKALGAPIEKTISKAK
GQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 6 is the amino acid sequence of the m276 variable light (VL)
domain. CDR sequences are indicated by bold underline.
EIVLTQSPATLSLSPGERATLSCRASQSVSSYLAWYQQKPGQAPRLLIYDASNRATGIPARF
SGSGSGTDFTLTISSLEPEDFAVYYCQQRSNWPPRITFGQGTRLEIK
SEQ ID NO: 7 is the amino acid sequence of the m276 light chain.
EIVLTQSPATLSLSPGERATLSCRASQSVSSYLAWYQQKPGQAPRLLIYDASNRATGIPARF
SGSGSGTDFTLTISSLEPEDFAVYYCQQRSNWPPRITFGQGTRLEIKRTVAAPSVFIFPPSDEQ
LKSGTASVVCLLNNFYPREAKVQWKVDNALQSGNSQESVTEQDSKDSTYSLSSTLTLSKA
DYEKHKVYACEVTHQGLSSPVTKSFNRGEC

### DETAILED DESCRIPTION

### I. Abbreviations

ADC antibody-drug conjugate
B7H3 B7 homolog 3
CDR complementarity determining region
Ig immunoglobulin
PBD pyrrolobenzodiazepine
PEG polyethylene glycol
VH variable heavy
VL variable light

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Administration: To provide or give to a subject an agent, for example, a composition that includes an ADC that specifically targets CD276, by any effective route. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and is functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8:e1002388, 2012; and Kunik et al., Nucleic Acids Res 40 (Web Server issue):W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online. In some embodiments herein, amino acid numbering (such as to define the location of an amino acid substitution) is referenced according to the Eu numbering convention (see Edelman et al., Proc. Natl. Acad. Sci. USA 63: 78-85, 1969).

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one embodiment, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Antibody-drug conjugate (ADC): A molecule that includes an antibody (or antigen-binding fragment of an antibody) conjugated to a drug, such as a cytotoxic agent. ADCs can be used to specifically target a drug to cancer cells through specific binding of the antibody to a tumor antigen expressed on the cell surface. Exemplary drugs for use with ADCs include anti-microtubule agents (such as maytansinoids, auristatin E and auristatin F) and interstrand crosslinking agents (for example, pyrrolobenzodiazepines; PBDs).

Anti-microtubule agent: A type of drug that blocks cell growth by stopping mitosis. Anti-microtubule agents, also referred to as “anti-mitotic agents,” are used to treat cancer.

Binding affinity: Affinity of an antibody for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In other embodiments, antibody affinity is measured by flow cytometry or by surface plasmon reference. An antibody that “specifically binds” an antigen (such as CD276) is an antibody that binds the antigen with high affinity and does not significantly bind other unrelated antigens.

Breast cancer: A type of cancer that forms in tissues of the breast, usually the ducts and lobules. Types of breast cancer include, for example, ductal carcinoma in situ, invasive ductal carcinoma, triple negative breast cancer, inflammatory breast cancer, metastatic breast cancer, medullary carcinoma, tubular carcinoma and mucinous carcinoma. Triple negative breast cancer refers to a type of breast cancer in which the cancer cells do not express estrogen receptors, progesterone receptors or significant levels of HER2/neu protein. Triple negative breast cancer is also called ER-negative PR-negative HER2/neu-negative breast cancer. Infiltrating (malignant) carcinoma of the breast can be divided into stages (I, IIA, IIB, IIIA, IIIB, and IV). See, for example, Bonadonna et al., (eds), “Textbook of Breast Cancer: A clinical Guide the Therapy,” 3^{rd}; London, Tayloy & Francis, 2006.

CD276: An immune checkpoint molecule that is expressed by some types of solid tumors. This protein is a member of the B7 superfamily of co-stimulatory molecules. CD276 is also known as B7 homolog 3 (B7-H3).

CD276-positive cancer: A cancer that expresses or overexpresses CD276. Examples of CD276-positive cancers include, but are not limited to, liver cancers (such as hepatocellular carcinoma), pancreatic cancers, kidney cancers, bladder cancers, cervical cancers, endometrial cancer, esophageal cancers, prostate cancers, breast cancers, ovarian cancers, colon cancers, lung cancers (such as non-small cell lung cancer), brain cancers (such as neuroblastoma or glioblastoma), pediatric cancers (such as osteosarcoma, neuroblastoma, rhabdomyosarcoma, Wilms tumor or Ewing's sarcoma), melanoma and mesothelioma (see, for example, Seaman et al., Cancer Cell 31(4):501-505, 2017).

Chemotherapeutic agent: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating a CD276-positive tumor. In one embodiment, a chemotherapeutic agent is a radioactive compound. Non-limiting examples of chemotherapeutic agents of use can be found in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. One example is the administration of an ADC that targets CD276 used in combination with a radioactive or chemical compound.

Colon cancer: A type of cancer that develops in the colon or the rectum. The most common type of colon cancer (also known as “colorectal cancer”) is colorectal adenocarcinoma, which accounts for approximately 95% of all colon cancers. Adenocarcinomas develop in the cells lining the inside of the colon and/or rectum. Other types of colorectal cancers include gastrointestinal carcinoid tumors, metastatic colorectal cancer, primary colorectal lymphoma (a type of non-Hodgkin's lymphoma), gastrointestinal stromal tumors (classified as a sarcoma and arising from interstitial cells of Cajal), leiomyosarcoma (arising from smooth muscle cells) and colorectal melanoma.

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. A single-domain antibody contains three CDRs, referred to herein as CDR1, CDR2 and CDR3.

Conservative variant: A protein containing conservative amino acid substitutions that do not substantially affect or decrease the affinity of a protein, such as an antibody to CD276. For example, a monoclonal antibody that specifically binds CD276 can include at most about 1, at most about 2, at most about 5, and most about 10, or at most about 15 conservative substitutions and specifically bind the CD276 polypeptide. The term “conservative variant” also includes the use of a substituted amino acid in place of an unsubstituted parent amino acid, provided that antibody specifically binds CD276. Non-conservative substitutions are those that reduce an activity or binding to CD276.

Conservative amino acid substitution tables provide functionally similar amino acids. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Contacting: Placement in direct physical association; includes both in solid and liquid form.

Cytotoxic agent: Any drug or compound that kills cells.

Cytotoxicity: The toxicity of a molecule, such as an immunotoxin, to the cells intended to be targeted, as opposed to the cells of the rest of an organism. In contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Drug: Any compound used to treat, ameliorate or prevent a disease or condition in a subject. In some embodiments herein, the drug is an anti-cancer agent, for example a cytotoxic agent, such as an anti-mitotic or anti-microtubule agent.

Epitope: An antigenic determinant. These are particular chemical groups or peptide sequences on a molecule that are antigenic (that elicit a specific immune response). An antibody specifically binds a particular antigenic epitope on a polypeptide, such as CD276.

Framework region: Amino acid sequences interposed between CDRs. Framework regions of an immunoglobulin molecule include variable light and variable heavy framework regions.

Fusion protein: A protein comprising at least a portion of two different (heterologous) proteins.

Heterologous: Originating from a separate genetic source or species.

IgG: A polypeptide belonging to the class or isotype of antibodies that are substantially encoded by a recognized immunoglobulin gamma gene. In humans, this class includes IgG₁, IgG₂, IgG₃, and IgG₄. In mice, this class includes IgG₁, IgG₂, IgG_{2b}, and IgG₃.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4⁺ response or a CD8⁺ response. In another embodiment, the response is a B cell response, and results in the production of specific antibodies.

Interstrand crosslinking agent: A type of cytotoxic drug capable of binding covalently between two strands of DNA, thereby preventing DNA replication and/or transcription.

Isolated: An “isolated” biological component, such as a nucleic acid, protein (including antibodies) or organelle, has been substantially separated or purified away from other biological components in the environment (such as a cell) in which the component naturally occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an antibody. The linkage can be either by chemical or recombinant means. “Chemical means” refers to a reaction between the antibody moiety and the effector molecule such that there is a covalent bond formed between the two molecules to form one molecule. The ADCs disclosed herein include a linker to join the antibody to the drug. In some embodiments, the linker includes a maleimide group, a PEG (such as PEG8) and a valine-alanine dipeptide.

Liver cancer: Any type of cancer occurring in liver tissue. The most common type of liver cancer is hepatocellular carcinoma (HCC), which develops in hepatocytes. Other types of liver cancer include cholangiocarcinoma, which develops in the bile ducts; liver angiosarcoma, which is a rare form of liver cancer that begins in the blood vessels of the liver, and hepatoblastoma, which is a very rare type of liver cancer found most often in children.

Lung cancer: Any cancer that forms in the lung. Most cancers that begin in the lung are carcinomas. The two primary types of lung carcinoma are small-cell lung carcinoma (SCLC) and non-small cell lung carcinoma (NSCLC). Subclasses of NSCLC include adenocarcinoma, squamous-cell carcinoma and large-cell carcinoma. Lung cancer is typically staged from I to IV; other classifications are also used, for example small-cell lung carcinoma can be classified as limited stage if it is confined to one half of the chest and within the scope of a single radiotherapy field; otherwise, it is extensive stage. See, for example, Hansen (ed.), Textbook of Lung Cancer, 2^{nd}, London: Informa Healthcare, 2008.

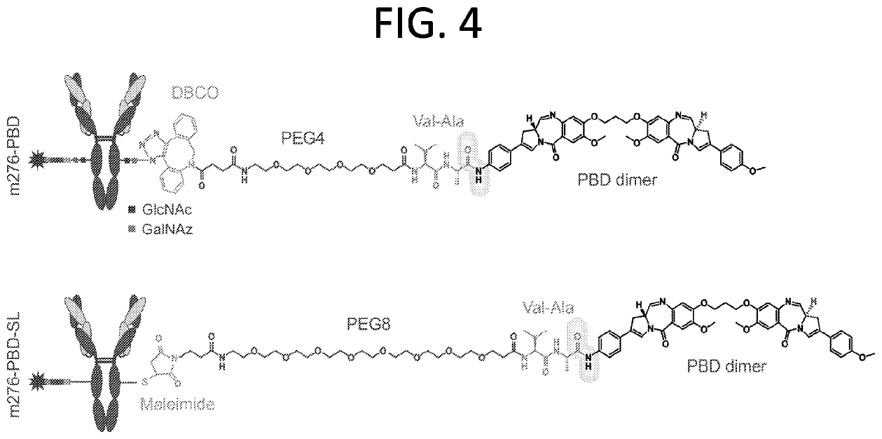

Maleimide: A chemical compound with the formula H₂C₂(CO)₂NH. Maleimide groups are commonly used for bioconjugation, such as for conjugation of a drug to an antibody (see, e.g., Ravasco et al., Chem Eur J 25: 43-49, 2019). Maleimides linked to polyethylene glycol (PEG) chains are often used as flexible linking molecules (see FIG. 4).

Neuroblastoma: A solid tumor arising from embryonic neural crest cells. Neuroblastoma commonly arises in and around the adrenal glands, but can occur anywhere that sympathetic neural tissue is found, such as in the abdomen, chest, neck or nerve tissue near the spine. Neuroblastoma typically occurs in children younger than 5 years of age.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Ovarian cancer: Cancer that forms in tissues of the ovary. Most ovarian cancers are either ovarian epithelial carcinomas (cancer that begins in the cells on the surface of the ovary) or malignant germ cell tumors (cancer that begins in egg cells). Another type of ovarian cancer is stromal cell cancer, which originates in cells that release hormones and connect the different structures of the ovaries.

Pancreatic cancer: A disease in which malignant cells are found in the tissues of the pancreas. Pancreatic tumors can be either exocrine tumors or neuroendocrine tumors, based on the cell origin of the cancer. The vast majority (˜94%) of pancreatic cancers are exocrine tumors. Exocrine cancers include, for example, adenocarcinoma (the most common type of exocrine tumor), acinar cell carcinoma, intraductal papillary-mucinous neoplasm (IPMN), and mucinous cystadenocarcinoma. In some examples, the pancreatic cancer is pancreatic ductal adenocarcinoma (PDAC). Pancreatic neuroendocrine tumors, also referred to as islet cell tumors, are classified by the type of hormones they produce. Exemplary neuroendocrine tumors include gastrinoma, glucaganoma, insulinoma, somatostatinoma, VIPoma (vasoactive intestinal peptide) and nonfunctional islet cell tumor.

Pediatric cancer: A cancer that develops in children ages 0 to 14. The major types of pediatric cancers include, for example, neuroblastoma, acute lymphoblastic leukemia (ALL), embryonal rhabdomyosarcoma (ERMS), alveolar rhabdomyosarcoma (ARMS), Ewing's sarcoma, desmoplastic small round cell tumor (DRCT), osteosarcoma, brain and other CNS tumors (such as neuroblastoma and medulloblastoma), Wilms tumor, non-Hodgkin lymphoma, and retinoblastoma.

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polyethylene glycol (PEG): A compound comprised of repeating ethylene oxide units with the chemical formula H—(O—CH₂—CH₂)ₙ—OH. PEG molecules are often used in linkers for ADCs due to their water solubility, lack of toxicity, low immunogenicity and well-defined chain lengths. PEG-containing linkers promote a decrease in protein aggregation and increase solubility of the conjugate. In some embodiments, the PEG of the ADC linker includes 4, 5, 6, 7, 8, 9 or 10 ethylene oxide units. In particular embodiments, the PEG of the ADC linker includes eight ethylene oxide units (PEG8).

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number of size of metastases. “Ameliorating” refers to the reduction in the number or severity of signs or symptoms of a disease, such as cancer.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Pyrrolobenzodiazepine (PBD): A class of sequence-selective DNA minor-groove binding crosslinking agents originally discovered in Streptomyces species. PBDs are significantly more potent than systemic chemotherapeutic drugs. The mechanism of action of PBDs is associated with their ability to form an adduct in the minor groove of DNA, thereby interfering with DNA processing. In the context of the present disclosure, PBDs include naturally produced and isolated PBDs, chemically synthesized naturally occurring PBDs, and chemically synthesized non-naturally occurring PBDs. PBDs also include monomeric, dimeric and hybrid PBDs (for a review see Gerratana, Med Res Rev 32(2):254-293, 2012).

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds a CD276 polypeptide are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. These sequence identity ranges are provided for guidance only; it is entirely possible that strongly significant homologs could be obtained that fall outside of the ranges provided.

Small molecule: A molecule, typically with a molecular weight less than about 1000 Daltons, or in some embodiments, less than about 500 Daltons, wherein the molecule is capable of modulating, to some measurable extent, an activity of a target molecule.

Subject: Living multi-cellular vertebrate organisms, a category that includes both human and veterinary subjects, including human and non-human mammals.

Synthetic: Produced by artificial means in a laboratory, for example a synthetic nucleic acid or protein (for example, an antibody) can be chemically synthesized in a laboratory.

Therapeutically effective amount: The amount of an agent (such as an ADC targeting CD276) that alone, or together with one or more additional agents, induces the desired response, such as, for example treatment of a tumor, in a subject. When administered to a subject, a dosage will generally be used that will achieve target tissue concentrations that has been shown to achieve a desired in vitro effect. Ideally, a therapeutically effective amount provides a therapeutic effect without causing a substantial cytotoxic effect in the subject.

In one example, a desired response is to decrease the size, volume, or number (such as metastases) of a tumor in a subject. For example, the agent or agents can decrease the size, volume, or number of tumors by a desired amount, for example by at least 5%, at least 10%, at least 15%, at least 20%, at least 25%, at least 30%, at least 50%, at least 75%, at least 90%, or at least 95% as compared to a response in the absence of the agent.

Several preparations disclosed herein are administered in therapeutically effective amounts. A therapeutically effective amount of an ADC that specifically binds CD276 (or a composition including an ADC) that is administered to a human or veterinary subject will vary depending upon a number of factors associated with that subject, for example the overall health of the subject. A therapeutically effective amount can be determined by varying the dosage and measuring the resulting therapeutic response, such as the regression of a tumor. Therapeutically effective amounts also can be determined through various in vitro, in vivo or in situ immunoassays. The disclosed agents can be administered in a single dose, or in several doses, as needed to obtain the desired response. However, the therapeutically effective amount of can be dependent on the source applied, the subject being treated, the severity and type of the condition being treated, and the manner of administration.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some embodiments, the vector is a virus vector, such as a lentivirus vector.

### III. Antibody-Drug Conjugates Targeting CD276

CD276-specific monoclonal antibody m276 (also known as m8524) was isolated from a naïve human scFv library, as previously described (WO 2016/044383, herein incorporated by reference in its entirety). The m276 antibody binds both human and mouse m276. WO 2016/044383 described two different ADCs using the m276 antibody—one ADC included m276 conjugated to monomethyl auristatin E (MMAE) via a linking moiety and the second ADC was comprised of m276 conjugated to pyrrolobenzodiazepine (PBD) via a glycol group (m276-PBD). Although the previously described ADCs were partially effective in killing CD276-expressing tumor cells, the present disclosure provides an improved CD276-specific ADC with enhanced stability and efficacy, and minimal off-target effects.

The improved CD276-targeted ADC includes several mutations in the Fc region of the m276 antibody. In particular, the ADC includes L234A, L235A and P329G substitutions (numbered with reference to human IgG1 according to Eu numbering convention) to render the Fc region non-reactive with FcγRI, FcγRII and FcγRIII, which reduces off-target effects by preventing killing of Fc receptor-expressing normal cells. The modified ADC also includes an S239C mutation (numbered with reference to human IgG1 according to Eu numbering convention) to allow for site-directed conjugation of a drug, such as PBD or a PBD dimer. As one example, the drug is conjugated to the cysteine at residue 239 using a valine-alanine dipeptide linker. Site-specific conjugation at this location improves the biophysical properties of the ADC by allowing conjugation of highly hydrophobic drugs (such as PBD) without significant aggregation. The S239C mutation also prevents the premature loss of the drug in the circulation, thereby enhancing stability of the ADC.

It is disclosed herein that a modified ADC containing antibody m276 and a PBD dimer as the drug component (referred to herein as “m276-PBD-SL”) is extremely potent in mouse tumor xenograft models. The m276-PBD-SL ADC is capable of eradicating very large tumors (>1000 mmn³), which was not possible with the original unmodified m276-based ADC. Furthermore, this effect was observed at doses of m276-PBD-SL that did not cause toxicity in the mice (see Examples 2 and 5).

The amino acid sequences of the m276 VH domain and VL domain are provided below; CDR sequences according to IMGT are indicated in bold underline. The amino acid residues of each CDR are listed below each sequence.

m276 variable heavy (VH) domain
(SEQ ID NO: 2)
QVQLQQSGAEVKKPGSSVKVSCKASGGTFSSYAISWVRQAPGQGLEWMG
GIIPILGIANYAQKFQGRVTITADESTSTAYMELSSLRSEDTAVYYCAR
GGSGSYHMDVWGKGTTVTVSS
CDR1 = residues 26-33
CDR2 = residues 51-58
CDR3 = residues 97-108
m276 variable light (VL) domain
(SEQ ID NO: 6)
EIVLTQSPATLSLSPGERATLSCRASQSVSSYLAWYQQKPGQAPRLLIY
DASNRATGIPARFSGSGSGTDFTLTISSLEPEDFAVYYCQQRSNWPPRIT
FGQGTRLEIK
CDR1 = residues 27-32
CDR2 = residues 50-52
CDR3 = residues 89-99

To generate a modified ADC using the m276 antibody, four amino acid substitutions were introduced into the heavy chain of m276. The sequence of the modified heavy chain is provided below and set forth herein as SEQ ID NO: 4. The four amino acid substitutions in the heavy chain, located at residues 236, 237, 241 and 331 of SEQ ID NO: 4, and corresponding to residues 234, 235, 239 and 329 of human IgG1, are shown in bold underline. The VH domain (residues 1-119 of SEQ ID NO: 4) is underlined. The constant region of the m276 heavy chain is set forth herein as SEQ ID NO: 3 (and corresponds to residues 120-449 of SEQ ID NO: 4).

Modified m276 heavy chain
(SEQ ID NO: 4)
QVQLQQSGAEVKKPGSSVKVSCKASGGTFSSYAISWVRQAPGQGLEWMG
GIIPILGIANYAQKFQGRVTITADESTSTAYMELSSLRSEDTAVYYCAR
GGSGSYHMDVWGKGTTVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPCVFLF
PPKPKDTLMISRTPEVTCVVVDVSHEDPEVKFNWYVDGVEVHNAKTKPR
EEQYNSTYRVVSVLTVLHQDWLNGKEYKCKVSNKALGAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK

In some embodiments, the modified m276 constant domain includes an N-terminal signal peptide: MEWSWVFLFFLSVTTGVHS (SEQ ID NO: 1). The modified heavy chain with the N-terminal signal sequence (underlined) is shown below, and is set forth herein as SEQ ID NO: 5.

Modified m276 heavy chain with signal sequence
(SEQ ID NO: 5)
MEWSWVFLFFLSVTTGVHSQVQLQQSGAEVKKPGSSVKVSCKASGGTFS
SYAISWVRQAPGQGLEWMGGIIPILGIANYAQKFQGRVTITADESTSTA
YMELSSLRSEDTAVYYCARGGSGSYHMDVWGKGTTVTVSSASTKGPSVF
PLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTH
TCPPCPAPEAAGGPCVFLFPPKPKDTLMISRTPEVTCVVVDVSHEDPEV
KFNWYVDGVEVHNAKTKPREEQYNSTYRVVSVLTVLHQDWLNGKEYKCK
VSNKALGAPIEKTISKAKGQPREPQVYTLPPSRDELTKNQVSLTCLVKG
FYPSDIAVEWESNGQPENNYKTTPPVLDSDGSFFLYSKLTVDKSRWQQG
NVFSCSVMHEALHNHYTQKSLSLSPGK

Provided herein are ADCs that include a drug conjugated to a CD276-specific monoclonal antibody. The monoclonal antibody includes a variable heavy (VH) domain, a variable light (VL) domain and an IgG1 Fc region. In some embodiments, the VH domain of the monoclonal antibody comprises the complementarity determining region 1 (CDR1), CDR2 and CDR3 sequences of the m276 VH domain (set forth as SEQ ID NO: 2), the VL domain of the monoclonal antibody comprises the CDR1, CDR2 and CDR3 sequences of the m276 VL domain (set forth as SEQ ID NO: 6), and the Fc region of the monoclonal antibody comprises S239C, L234A, L235A and P329G3 mutations (numbered with reference to human IgG1). The drug component of the ADC is conjugated (directly, or indirectly via a linker) to the cysteine at residue 239 of the Fc domain by site directed conjugation.

In some embodiments, the CDR sequences are determined using the Kabat, IMGT or Chothia numbering convention.

In some embodiments, the VH domain CDR1, CDR2 and CDR3 sequences respectively comprise residues 26-33, 51-58 and 97-108 of SEQ ID NO: 2; and/or the VL domain CDR1, CDR2 and CDR3 sequences respectively comprise residues 27-32, 50-52 and 89-99 of SEQ ID NO: 6. In some examples, the VH domain (in addition to the recited CDR sequences) comprises an amino acid sequence at least 80%, at least 85%, at least 90%, at least 91%, at least 92%, at least 93%, at least 94%, at least 95%, at least 96%, at least 97%, at least 98% or at least 99% identical to SEQ ID NO: 2 and/or the VL domain (in addition to the recited CDR sequences) comprises an amino acid sequence at least 80%, at least 85%, at least 90%, at least 91%, at least 92%, at least 93%, at least 94%, at least 95%, at least 96%, at least 97%, at least 98% or at least 99% identical to SEQ ID NO: 6. In particular non-limiting examples, the VH domain comprises or consists of the amino acid sequence of SEQ ID NO: 2 and/or the VL domain comprises or consists of the amino acid sequence of SEQ ID NO: 6.

In some embodiments, the amino acid sequence of the Fc region comprises SEQ ID NO: 3.

In some embodiments, the monoclonal antibody is an IgG1. In some examples, the monoclonal antibody is an IgG1 and the amino acid sequence of the heavy chain of the IgG1 comprises SEQ ID NO: 4 or SEQ ID NO: 5. In some examples, the monoclonal antibody is an IgG1 and the amino acid sequence of the light chain of the IgG1 comprises SEQ ID NO: 7.

In some embodiments, the drug of the ADC includes a cytotoxic agent, such as an interstrand crosslinking agent, an anti-mitotic agent or an anti-microtubule agent. In some examples, the interstrand crosslinking agent comprises a pyrrolobenzodiazepine (PBD), such as a PBD dimer.

In some embodiments, the ADC further includes a linker connecting the drug to the monoclonal antibody. In some examples, the drug is conjugated to the monoclonal antibody via a linker comprising a valine-alanine dipeptide. In specific examples, the linker further includes a maleimide group. In specific examples, the linker further includes a polyethylene glycol (PEG). In specific non-limiting examples, the linker includes a valine-alanine dipeptide, a PEG (such as PEG8), and a maleimide group.

Also provided herein are compositions that include an ADC as disclosed herein and a pharmaceutically acceptable carrier.

Further provided herein are methods of treating a CD276-positive cancer in a subject. In some embodiments, the method includes administering to the subject a therapeutically effective amount of an ADC or composition disclosed herein. In some examples, the method further includes selecting a subject diagnosed with a CD276-positive cancer. In some examples, the CD276-positive cancer is hepatocellular carcinoma, melanoma, leukemia, breast cancer, neuroblastoma, prostate cancer, colorectal cancer, osteosarcoma, endometrial cancer, ovarian cancer, oral squamous cell carcinoma, non-small cell lung cancer, bladder cancer or pancreatic cancer. In specific non-limiting examples, the CD276-positive cancer is breast cancer or neuroblastoma.

Also provided are methods of inhibiting tumor growth or metastasis of a CD276-positive cancer in a subject. In some embodiments, the method includes administering to the subject a therapeutically effective amount of an ADC or composition disclosed herein. In some examples, the method further includes selecting a subject diagnosed with a CD276-positive cancer. In some examples, the CD276-positive cancer is hepatocellular carcinoma, melanoma, leukemia, breast cancer, neuroblastoma, prostate cancer, colorectal cancer, osteosarcoma, endometrial cancer, ovarian cancer, oral squamous cell carcinoma, non-small cell lung cancer, bladder cancer or pancreatic cancer. In specific non-limiting examples, the CD276-positive cancer is breast cancer or neuroblastoma.

In some embodiments of the disclosed methods, the method further includes administering to the subject an additional anti-cancer agent. In some examples, the additional anti-cancer agent comprises a chemotherapeutic agent or an anti-angiogenesis agent. In some embodiments, the method further includes surgical resection of a tumor and/or radiation therapy.

### IV. Drugs and Linkers

ADCs are compounds comprised of a tumor antigen-specific antibody and a drug, typically a cytotoxic agent, such as an anti-microtubule agent or cross-linking agent. Because ADCs are capable of specifically targeting cancer cells, the drug can be much more potent than agents used for standard chemotherapy. The most common cytotoxic drugs currently used with ADCs have an IC₅₀ that is 100- to 1000-fold more potent than conventional chemotherapeutic agents. Common cytotoxic drugs include pyrrolobenzodiazepines (PDBs), which covalently bind the minor groove of DNA to form interstrand crosslinks, and anti-microtubule agents, such as maytansinoids and auristatins (such as auristatin E and auristatin F). In some instances, ADCs comprise a 1:2 to 1:4 ratio of antibody to drug (Bander, Clinical Advances in Hematology & Oncology 10(8; suppl 10):3-7, 2012).

Provided herein are ADCs that include a drug (such as a cytotoxic agent) conjugated to a monoclonal antibody that binds (such as specifically binds) CD276. In some embodiments, the drug is a small molecule. In some examples, the drug is a cross-linking agent, an anti-microtubule agent and/or anti-mitotic agent, or any cytotoxic agent suitable for mediating killing of tumor cells. Exemplary cytotoxic agents include, but are not limited to, a PDB, an auristatin, a maytansinoid, dolastatin, calicheamicin, nemorubicin and its derivatives, PNU-159682, anthracycline, duocarmycin, vinca alkaloid, taxane, trichothecene, CC1065, camptothecin, elinafide, a combretastain, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity.

In some embodiments, the ADC comprises a pyrrolobenzodiazepine (PBD). The natural product anthramycin (a PBD) was first reported in 1965 (Leimgruber et al., J Am Chem Soc, 87:5793-5795, 1965; Leimgruber et al., J Am Chem Soc, 87:5791-5793, 1965). Since then, a number of PBDs, both naturally-occurring and synthetic analogues, have been reported (Gerratana, Med Res Rev 32(2):254-293, 2012; and U.S. Pat. Nos. 6,884,799; 7,049,311; 7,067,511; 7,265,105; 7,511,032; 7,528,126; and 7,557,099). As one example, PDB dimers recognize and bind to specific DNA sequences, and are useful as cytotoxic agents. PBD dimers have been conjugated to antibodies and the resulting ADC had anti-cancer properties (see, for example, US 2010/0203007). Exemplary linkage sites on the PBD dimer include the five-membered pyrrole ring, the tether between the PBD units, and the N10-C11 imine group (see WO 2009/016516; US 2009/304710; US 2010/047257; US 2009/036431; US 2011/0256157; and WO 2011/130598).

In some embodiments, the ADC includes an antibody conjugated to one or more maytansinoid molecules. Maytansinoids are derivatives of maytansine, and are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

In some embodiments, the ADC includes an antibody conjugated to a dolastatin or auristatin, or an analog or derivative thereof (see U.S. Pat. Nos. 5,635,483; 5,780,588; 5,767,237; and 6,124,431). Auristatins are derivatives of the marine mollusk compound dolastatin-10. Dolastatins and auristatins interfere with microtubule dynamics, GTP hydrolysis, and nuclear and cellular division (Woyke et al., Antimicrob Agents and Chemother 45(12):3580-3584, 2001) and have anticancer (U.S. Pat. No. 5,663,149) and antifungal activity (Pettit et al., Antimicrob Agents Chemother 42:2961-2965, 1998). Exemplary dolastatins and auristatins include, but are not limited to, dolastatin 10, auristatin E, auristatin F, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), and other auristatins (see, for example, U.S. Publication No. 2013/0129753).

In some embodiments, the ADC includes an antibody conjugated to one or more calicheamicin molecules. The calicheamicin family of antibiotics, and analogues thereof, are capable of producing double-stranded DNA breaks at sub-picomolar concentrations (Hinman et al., Cancer Res 53:3336-3342, 1993; Lode et al., Cancer Res 58:2925-2928, 1998). Exemplary methods for preparing ADCs with a calicheamicin drug moiety are described in U.S. Pat. Nos. 5,712,374; 5,714,586; 5,739,116; and 5,767,285.

In some embodiments, the ADC includes an anthracycline. Anthracyclines are antibiotic compounds that exhibit cytotoxic activity. It is believed that anthracyclines can operate to kill cells by a number of different mechanisms, including intercalation of the drug molecules into the DNA of the cell thereby inhibiting DNA-dependent nucleic acid synthesis; inducing production of free radicals which then react with cellular macromolecules to cause damage to the cells; and/or interactions of the drug molecules with the cell membrane. Non-limiting exemplary anthracyclines include doxorubicin, epirubicin, idarubicin, daunomycin, daunorubicin, doxorubicin, epirubicin, nemorubicin, valrubicin and mitoxantrone, and derivatives thereof. For example, PNU-159682 is a potent metabolite (or derivative) of nemorubicin (Quintieri et al., Clin Cancer Res 11(4):1608-1617, 2005). Nemorubicin is a semisynthetic analog of doxorubicin with a 2-methoxymorpholino group on the glycoside amino of doxorubicin (Grandi et al., Cancer Treat Rev 17:133, 1990; Ripamonti et al., Br J Cancer 65:703-707, 1992).

The antibody and drug can be linked by a cleavable or non-cleavable linker. However, in some instances, it is desirable to have a linker that is stable in the circulation to prevent systemic release of the cytotoxic drug that could result in significant off-target toxicity. Non-cleavable linkers prevent release of the cytotoxic agent before the ADC is internalized by the target cell. Once in the lysosome, digestion of the antibody by lysosomal proteases results in the release of the cytotoxic agent (Bander, Clinical Advances in Hematology & Oncology 10(8; suppl 10):3-7, 2012).

In some embodiments, the linker has a functionality that is capable of reacting with a free cysteine present on an antibody to form a covalent bond. Exemplary linkers with such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some examples herein, the linker is non-cleavable and is directly conjugated to the antibody by site-specific conjugation. In some examples, the linker of the ADC includes a valine-alanine dipeptide, a PEG molecule, and a maleimide group.

### V. Compositions and Methods of Use

Compositions are provided that include a CD276-specific ADC disclosed herein. The compositions can be prepared in unit dosage form for administration to a subject. The amount and timing of administration are at the discretion of the treating clinician to achieve the desired outcome. The ADC can be formulated for systemic or local (such as intra-tumor) administration. In one example, the ADC is formulated for parenteral administration, such as intravenous administration.

The compositions for administration can include a solution of the ADC in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of ADC in these formulations can vary widely, and will be selected primarily based on fluid volumes, viscosities, body weight and the like in accordance with the particular mode of administration selected and the subject's needs.

The compositions that include an ADC can be formulated in unit dosage form suitable for individual administration of precise dosages. In addition, the compositions may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

Typical dosages of the ADCs, compositions or additional agents can range from about 0.01 to about 30 mg/kg, such as from about 0.1 to about 10 mg/kg. In some examples, the dosage is at least about 0.1 mg/kg, at least about 0.2 mg/kg, at least about 0.3 mg/kg, at least about 0.4 mg/kg, at least about 0.5 mg/kg, at least about 1 mg/kg, at least about 4 mg/kg, at least about 3 mg/kg, at least about 5 mg/kg, at least about 6 mg/kg, at least about 7 mg/kg, at least about 8 mg/kg is at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, at least about 20 mg/kg, at least about 21 mg/kg, at least about 22 mg/kg, at least about 23 mg/kg, at least about 24 mg/kg at least about 25 mg/kg, at least about 26 mg/kg, at least about 27 mg/kg, at least about 28 mg/kg, at least about 29 mg/kg, or at least about 30 mg/kg.

In particular examples, the subject is administered an ADC or composition thereof, or additional agent(s), on a multiple daily dosing schedule, such as at least two consecutive days, 10 consecutive days, and so forth, for example for a period of weeks, months, or years. In one example, the subject is administered the ADC, composition or additional agent(s) for a period of at least 30 days, such as at least 2 months, at least 4 months, at least 6 months, at least 12 months, at least 24 months, or at least 36 months.

In some embodiments, a disclosed ADC or composition is administered intravenously, subcutaneously or by another mode daily or multiple times per week for a period of time, followed by a period of no treatment, then the cycle is repeated. In some embodiments, the initial period of treatment (e.g., administration of the therapeutic agent daily or multiple times per week) is for 3 days, 1 week, 2 weeks, 3 weeks, 4 weeks, 5 weeks, 6 weeks, 7 weeks, 8 weeks, 9 weeks, 10 weeks, 11 weeks or 12 weeks. In a related embodiment, the period of no treatment lasts for 3 days, 1 week, 2 weeks, 3 weeks or 4 weeks. In certain embodiments, the dosing regimen of the therapeutic agent is daily for 3 days followed by 3 days off; or daily or multiple times per week for 1 week followed by 3 days or 1 week off; or daily or multiple times per week for 2 weeks followed by 1 or 2 weeks off; or daily or multiple times per week for 3 weeks followed by 1, 2 or 3 weeks off; or daily or multiple times per week for 4, 5, 6, 7, 8, 9, 10, 11 or 12 weeks followed by 1, 2, 3 or 4 weeks off.

The ADCs disclosed herein can also be administered by other routes, including via inhalation, oral, topical or intraocular. In some examples, the ADC is administered via fine-needle.

ADCs may be provided in lyophilized form and rehydrated with sterile water before administration, although they are also provided in sterile solutions of known concentration. The ADC solution is then added to an infusion bag containing 0.9% sodium chloride, USP, and in some cases administered at a dosage of from 0.5 to 15 mg/kg of body weight. Considerable experience is available in the art in the administration of antibody drugs, which have been marketed in the U.S. since the approval of RITUXAN™ in 1997. ADCs can be administered by slow infusion, rather than in an intravenous push or bolus. In one example, a higher loading dose is administered, with subsequent, maintenance doses being administered at a lower level.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include, for example, microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the ADC compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It is an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

The ADCs disclosed herein can be administered to slow or inhibit the growth of tumor cells or inhibit the metastasis of tumor cells, such as CD276-positive tumors, such as solid tumors. In these applications, a therapeutically effective amount of a composition is administered to a subject in an amount sufficient to inhibit growth, replication or metastasis of cancer cells, or to inhibit a sign or a symptom of the cancer. Suitable subjects may include those diagnosed with a cancer that expresses CD276, such as, but not limited to hepatocellular carcinoma, melanoma, leukemia, breast cancer, neuroblastoma, prostate cancer, colorectal cancer, osteosarcoma, endometrial cancer, ovarian cancer, oral squamous cell carcinoma, non-small cell lung cancer, bladder cancer or pancreatic cancer.

Provided herein is a method of treating a CD276-positive cancer in a subject by administering to the subject a therapeutically effective amount of an ADC or composition disclosed herein. Also provided herein is a method of inhibiting tumor growth or metastasis of a CD276-positive cancer in a subject by administering to the subject a therapeutically effective amount of an ADC or composition disclosed herein. In some embodiments, the CD276-positive cancer is hepatocellular carcinoma, melanoma, leukemia, breast cancer, neuroblastoma, prostate cancer, colorectal cancer, osteosarcoma, endometrial cancer, ovarian cancer, oral squamous cell carcinoma, non-small cell lung cancer, bladder cancer or pancreatic cancer.

A therapeutically effective amount of a CD276-specific ADC or composition disclosed herein will depend upon the severity of the disease, the type of disease, and the general state of the patient's health. A therapeutically effective amount of the antibody-based composition is that which provides either subjective relief of a symptom(s) or an objectively identifiable improvement as noted by the clinician or other qualified observer.

Administration of the CD276-specific ADCs and compositions disclosed herein can also be accompanied by administration of other anti-cancer agents or therapeutic treatments (such as surgical resection of a tumor). Any suitable anti-cancer agent can be administered in combination with the ADCs and compositions disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

Non-limiting examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Non-limiting examples of antimetabolites include folic acid analogs (such as methotrexate), pyrimidine analogs (such as 5-FU or cytarabine), and purine analogs, such as mercaptopurine or thioguanine.

Non-limiting examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

Non-limiting examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Non-limiting examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone). Examples of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Another common treatment for some types of cancer is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

The following examples are provided to illustrate certain particular features and/or embodiments. These examples should not be construed to limit the disclosure to the particular features or embodiments described.

### EXAMPLES

### Example 1: Modification of the Heavy Chain of CD276-Specific Antibody m276

Human CD276-specific antibody m276 (also known as “m8524”) was selected from a yeast display naïve human antibody library, as described in PCT Publication No. WO 2016/044383, which is herein incorporated by reference in its entirety. Since the m276 variable domains were derived from a natural (non-synthetic) human antibody library, the m276 IgG antibody, when administered into humans, will have a low probability of being recognized by the immune system. Most, if not all, previously described CD276 antibodies were originally developed using traditional hybridoma technology in mice. Consequently, those murine antibodies, even following humanization, still contain murine variable domains and will therefore still be more foreign (and therefore more immunogenic) than m276, which has fully-human variable domains.

The m276 antibody binds to both human and mouse CD276. The amino acid sequence of the VH domain and VL domain of m276 are set forth herein as SEQ ID NO: 2 and SEQ ID NO: 6, respectively.

This example describes the generation of a modified version of m276 IgG1. Specifically, four amino acid substitutions were incorporated into the heavy chain constant region of m276: L234A, L235A, P329G and S239C (numbered according to the Eu numbering convention for human IgG1; Edelman et al., Proc. Natl. Acad. Sci. USA 63: 78-85, 1969). The sequence of the modified m276 heavy chain is shown below (and set forth as SEQ ID NO: 5):

MEWSWVFLFFLSVTTGVHSQVQLQQSGAEVKKPGSSVKVSCKASGGTFS
SYAISWVRQAPGQGLEWMGGIIPILGIANYAQKFQGRVTITADESTSTA
YMELSSLRSEDTAVYYCARGGSGSYHMDVWGKGTTVTVSSASTKGPSVF
PLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTH
TCPPCPAPEAAGGPCVFLFPPKPKDTLMISRTPEVTCVVVDVSHEDPEV
KFNWYVDGVEVHNAKTKPREEQYNSTYRVVSVLTVLHQDWLNGKEYKCK
VSNKALGAPIEKTISKAKGQPREPQVYTLPPSRDELTKNQVSLTCLVKG
FYPSDIAVEWESNGQPENNYKTTPPVLDSDGSFFLYSKLTVDKSRWQQG
NVFSCSVMHEALHNHYTQKSLSLSPGK

The heavy chain sequence includes a 19-amino acid signal sequence (in italics above; SEQ ID NO: 1), the m276 VH domain (underlined above; SEQ ID NO: 2), and the m276 heavy chain constant domain (SEQ ID NO: 3); the four amino acid substitutions are indicated by bold underline.

The L234A, L235A and P329G (“LALAPG”) mutations were introduced to prevent the interaction of the Fc domain with endogenous Fc receptors present on cells of the reticuloendothelial system (Lo et al., J Biol Chem 292(9):3900-3908, 2017). These three mutations render the Fc region unreactive with Fcγ receptor I (RI), FcγRII and FcγRIII. Although many groups have attempted to enhance Fc/Fc receptor interactions to improve the ADCC or CDC activity of unarmed antibodies (i.e. antibodies without a drug conjugate), in the case of toxic ADCs, such interactions can become a liability if the internalized antibody kills the phagocytic target cell. Therefore, blocking ADC-Fc/Fc receptor interactions can prevent the inappropriate killing of Fc receptor-bearing normal cells, minimizing off-target toxicity.

The cysteine introduced at position 239 is used for attachment of the drug (a PBD dimer in this example). The resulting ADC is referred to as “m276-PBD-SL” (SL stands for S239C, LALAPG mutations). The engineered cysteine residue allows for site directed conjugation of the drug. The site-specific labeling of antibodies through the introduction of surface cysteines residues is described in Lyons et al. (Protein Eng 3(8):703-708, 1990). PBD payload attachment at the S239C site is important for multiple reasons (Jeffrey et al., Bioconjug Chem 24(7):1256-63, 2013). First, attachment at this site improves the biophysical properties of the ADC. PBD drugs are extremely hydrophobic, which makes it difficult to conjugate them to antibodies without causing the antibodies to aggregate. Antibody aggregation is a major concern in the ADC field because aggregated antibodies have unpredictable biophysical properties and can, for example, precipitate out of solution, or bind non-specifically to non-target cells. Conjugation at this site (S239C) increases the solubility of the ADC, drastically reducing its tendency to aggregate. Second, the S239C site protects the conjugated drug from falling off the antibody in the presence of scavenging sulfhydryls in serum, such as cysteine-34 in albumin, through a so-called retroMichael reaction (Sussman et al., Protein Eng Des Sel 31(2):47-54, 2018). Finally, the S239C site of conjugation also prevents the premature cleavage of the valine-alanine dipeptide by circulating enzymes, which can also result in premature shedding of the drug in serum, thereby enhancing stability of the ADC. The m276-PBD ADC described in WO 2016/044383 was less stable than the presently disclosed m276-PBD-SL ADC because PBD was conjugated to the antibody via a glycol group (see FIG. 4), directly exposing the dipeptide linker to serum proteases.

### Example 2: Treatment with m276-PBD-SL Provides Potent Anti-Tumor Activity in Animal Tumor Models

This example demonstrates that m276-PBD-SL is capable of eradicating large tumors in mouse models of human neuroblastoma and breast cancer.

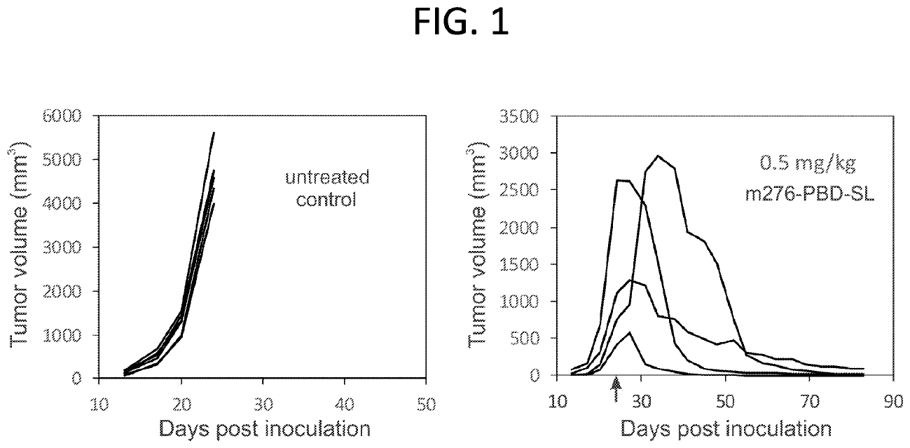

The m276-PBD-SL ADC was tested in a human NB-EB neuroblastoma xenograft model grown subcutaneously in mice. In this study, treatment with m276-PBD-SL was initiated when tumors reached an average size of approximately 1200 mm³. Animals were administered 0.5 mg/kg m276-PBD-SL (N=4) once per week starting on day 24 post-inoculation with tumor cells. Untreated animals were used as controls (N=6). The results demonstrated that m276-PBD-SL elicited potent antitumor activity against the neuroblastoma xenograft tumors (FIG. 1).

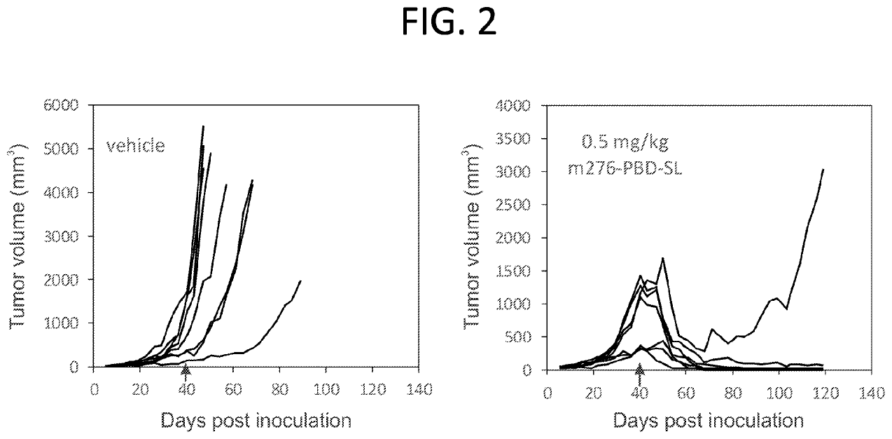

The m276-PBD-SL ADC was evaluated in a second model of human neuroblastoma called IMR-5. Treatment with vehicle (N=8) or m276-PBD-SL (N=7) was initiated when tumors reached an average size of approximately 1000 mm³. Animals were administered vehicle or 0.5 mg/kg m276-PBD-SL once per week starting on day 40 post-inoculation of tumor cells. The results of this study demonstrated that m276-PBD-SL elicited potent antitumor activity against the human neuroblastoma xenograft tumors grown subcutaneously in mice (FIG. 2).

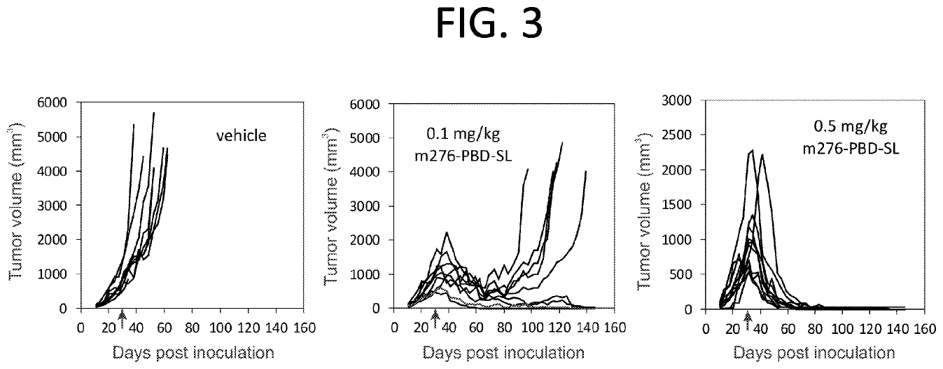

In another study, the m276-PBD-SL ADC was tested in a human MDA-MB-231 breast xenograft tumor model grown orthotopically in mice. Treatment with vehicle (N=10) or m276-PBD-SL (N=11) was initiated when tumors reached an average size of approximately 1000 mm³. Animals were administered vehicle, 0.1 mg/kg m276-PBD-SL or 0.5 mg/kg m276-PBD-SL once per week starting on day 31 after inoculation with tumor cells. As shown in FIG. 3, m276-PBD-SL elicited potent antitumor activity in this model of human breast cancer.

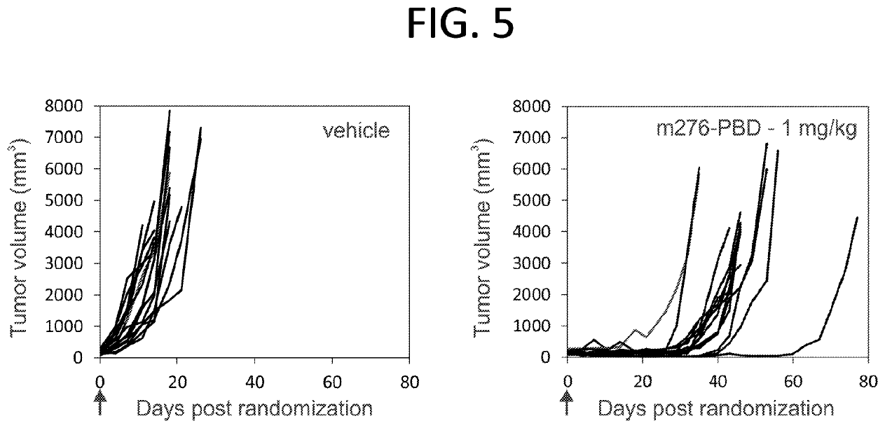

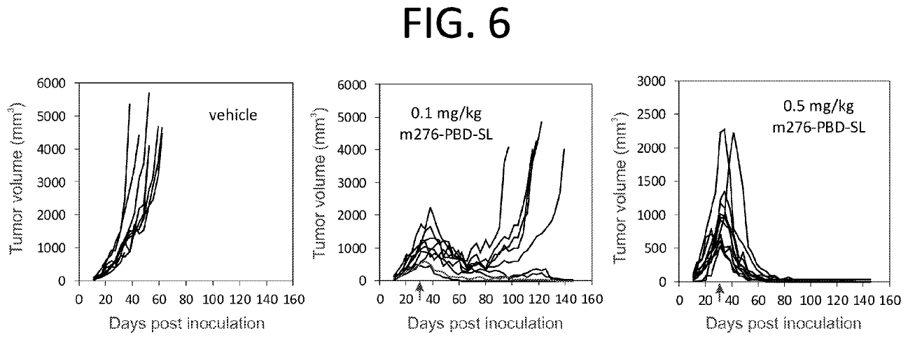

Additional studies were performed to compare the efficacy of the previously described m276-PBD glycoconjugate (WO 2016/044383) to the m276-PBD-SL ADC disclosed herein. In the first study, m276-PBD was tested in an orthotopic Py230 breast cancer model. Mice bearing Py230 tumors were administered vehicle or 1 mg/kg m276-PBD twice per week for four weeks. Treatment was initiated when the average tumor volume reached 140 mm³. As shown in FIG. 5, breast tumors in all mice treated with m276-PBD glycoconjugate initially regressed and then relapsed after treatment. As a comparison, the m276-PBD-SL ADC was evaluated in a large orthotopic MDA-MB-231 breast cancer model. Treatment was initiated when the average tumor volume reached 1000 mm³. Mice bearing MDA-MB-231 tumors were administered vehicle, 0.1 mg/kg m276-PBD-SL or 0.5 mg/kg m276-PBD-SL once per week for five weeks. As shown in FIG. 6, relapse occurred in some of the mice treated with the lower (0.1 mg/kg) dose, but complete responses were observed in all mice treated with the higher (0.5 mg/kg) dose of m276-PBD-SL. Thus, a lower dose of m276-PBD-SL was able to successfully treat larger tumors than a higher dose of the m276-PBD glycoconjugate was able to treat smaller tumors.

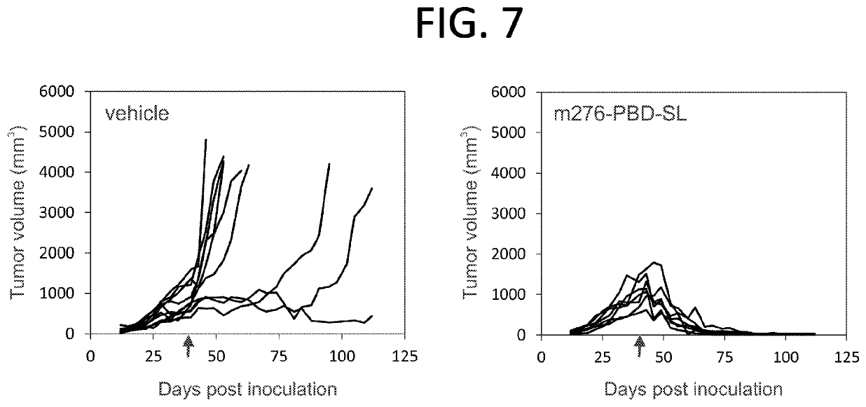

The m276-PBD-SL ADC was further tested in an orthotopic SUM519 breast cancer model. Mice bearing SUM159 tumors were administered vehicle or 0.5 mg/kg m276-PBD-SL (right) once per week for four weeks. Treatment was initiated when the average tumor volume reached 1000 mm³. The results demonstrated that treatment with m276-PBD-SL led to complete regression of SUM159 tumors (FIG. 7).

These results demonstrate that the m276-PBD-SL ADC is extremely potent, eradicating tumors at drug doses that show no sign of toxicity in mice. The drug is so potent that it is able to eradicate large tumors that are 1000 mm³ or greater in size. This level of potency is extremely unusual for any preclinical drug, and was not observed with the m276-PBD glycoconjugate.

### Example 3: Evaluation of m276-PBD-SL in a Mouse Model of UACC-62 Human Melanoma

This example describes a study to compare the effectiveness of m276-PBD-SL to the m276 PBD glycoconjugate ADC in a mouse model of UACC-62 human melanoma.

Mice bearing UACC-62 melanoma tumors are administered vehicle, 0.1 mg/kg m276-PBD, 0.5 mg/kg m276-PBD, 0.1 mg/kg m276-PBD-SL, or 0.5 mg/kg m276-PBD-SL once per week for 4 weeks, five weeks or six weeks. It is expected that treatment with m276-PBD-SL will result in complete or significant eradication of tumors. It is also expected that m276-PBD-SL will be significantly more effective that the m276-PDB glycoconjugate.

### Example 4: Evaluation of m276-PBD-SL in a Mouse Model of HCT-116 Human Colon Carcinoma

This example describes a study to compare the effectiveness of m276-PBD-SL to the m276 PBD glycoconjugate ADC in a mouse model of HCT-116 human colon carcinoma.

Mice bearing HCT-116 colon carcinoma tumors are administered vehicle, 0.1 mg/kg m276-PBD, 0.5 mg/kg m276-PBD, 0.1 mg/kg m276-PBD-SL, or 0.5 mg/kg m276-PBD-SL once per week for 4 weeks, five weeks or six weeks. It is expected that treatment with m276-PBD-SL will result in complete or significant eradication of tumors. It is also expected that m276-PBD-SL will be significantly more effective that the m276-PDB glycoconjugate.

### Example 5: Evaluation of m276-PBD-SL in Precinical Models of Pediatric Cancers

This example describes anti-tumor activity of m276-PBD-SL against preclinical xenograft models of pediatric solid tumors.

### Methods

Antibody conjugate m276-PBD-SL was tested in subcutaneous mouse xenograft models of Ewing sarcoma, rhabdomyosarcoma, Wilms tumor, osteosarcoma and neuroblastoma. m276-PBD-SL was administered by intraperitoneal injection at a dose of 0.5 mg/kg, once weekly for three consecutive weeks. Events were defined as a 4-fold increase in tumor volume from the first day of treatment. The Kaplan-Meier method was used to compare time-to-event between treated and control groups. The objective response categories are described as follows (see also, Houghton et al., Pediatr Blood Cancer 49(7):928-940, 2007):
PD=progressive disease, <50% tumor regression throughout study and >25% tumor growth at end of study
PD1=when PD and the mouse's time to event ≤200% the KM median time-to-event in control group
PD2=when PD but, additionally, time-to-event is >200% of the Kaplan-Meier (KM) median time-to-event in control group
SD=stable disease, <50% tumor regression throughout study and ≤25% tumor growth at end of study
PR=partial response, ≥50% tumor regression at any point during study but measurable tumor throughout study period
CR=complete response, disappearance of measurable tumor mass during study period
MCR=maintained complete response, no measurable tumor mass for at least 3 consecutive weekly readings at any time after treatment has been completed

Neuroblastoma testing used two animals per model to evaluate for tumor regression, while the other histologies used standard testing procedures (n=8-10) to evaluate for tumor regression and for time to event.

### CD276 Expression in Pediatric Preclinical Testing Consortium (PPTC) Models

CD276 mRNA expression was evaluated in PPTC models using RNA-Seq, measured in fragments per kilobase million (FPKM). The results demonstrated that CD276 expression was highest in solid tumors (median 41 FPKM), with the highest expression observed in osteosarcoma (median 82 FPKM). Neuroblastoma, rhabdomyosarcoma, Wilms tumor and embryonal brain tumor models also had elevated levels of expression, whereas acute lymphoblastic leukemia (ALL) models exhibited low levels of expression. The RNA-Seq data is consistent with protein expression data from clinical specimens (Majzner et al., Clin Cancer Res 25(8):2560-2574, 2019

### Summary of Tumor Growth Results

m276-PBD-SL showed very high levels of anti-tumor activity against several pediatric solid tumor preclinical models at a dose of 0.5 mg/kg, administered weekly for three weeks. Objective responses (PR/CR/MCR) were observed in 23 of 25 models (92%), including complete response (CR)/maintained complete response (MCR) in 4/5 osteosarcoma, 4/4 rhabdomyosarcoma, 3/3 Ewing sarcoma, 2/2 Wilms tumor, and 6/11 neuroblastoma models. The duration of response was prolonged after the final day of treatment (day 15), with most models that attained CR not showing regrowth by day 56.

There was no clear relationship between CD276 mRNA expression as measured by RNA-Seq and the response to m276-PBD-SL, with CR and MCR observed in models with CD276 expression ranging from 20 to 166 FPKM, and stable disease (SD)/partial response (PR) observed at expression levels from 14 to 131 FPKM.

Additionally, m276-PBD-SL was well tolerated, as evidenced by a toxic death rate of less than 2% and a mean body weight loss of 9.5%.

In view of the many possible embodiments to which the principles of the disclosed subject matter may be applied, it should be recognized that the illustrated embodiments are only preferred examples of the disclosure and should not be taken as limiting the scope of the disclosure. Rather, the scope of the disclosure is defined by the following claims. We therefore claim all that comes within the scope and spirit of these claims.

1. An antibody-drug conjugate (ADC), comprising a drug conjugated to a monoclonal antibody that specifically binds CD276, wherein:
the monoclonal antibody comprises a variable heavy (VH) domain, a variable light (VL) domain and an IgG1 Fc region, wherein the VH domain comprises the complementarity determining region 1 (CDR1), CDR2 and CDR3 sequences of SEQ ID NO: 2, the VL domain comprises the CDR1, CDR2 and CDR3 sequences of SEQ ID NO: 6, and the Fc region comprises S239C, L234A, L235A and P329G mutations according to the Eu numbering convention; and
the drug is conjugated to the cysteine at residue 239 of the Fc domain by site directed conjugation.

2. The ADC of claim 1, wherein the CDR sequences are determined using the Kabat, IMGT or Chothia numbering convention.

3. The ADC of claim 1, wherein the VH domain CDR1, CDR2 and CDR3 sequences respectively comprise residues 26-33, 51-58 and 97-108 of SEQ ID NO: 2 and the VL domain CDR1, CDR2 and CDR3 sequences respectively comprise residues 27-32, 50-52 and 89-99 of SEQ ID NO: 6.

4. The ADC of claim 1, wherein the VH domain comprises the amino acid sequence of SEQ ID NO: 2 and the VL domain comprises the amino acid sequence of SEQ ID NO: 6.

5. The ADC of claim 1, wherein the amino acid sequence of the Fc region comprises SEQ ID NO: 3.

6. The ADC of claim 1, wherein the monoclonal antibody is an IgG1.

7. The ADC of claim 6, wherein the amino acid sequence of the heavy chain of the IgG1 comprises or consists of SEQ ID NO: 4.

8. The ADC of claim 6, wherein the amino acid sequence of the light chain of the IgG1 comprises or consists of SEQ ID NO: 7.

9. The ADC of claim 1, wherein the drug is a dimer of pyrrolobenzodiazepine (PBD).

10. The ADC of claim 1, wherein the drug is conjugated to the monoclonal antibody via a linker comprising a valine-alanine dipeptide.

11. The ADC of claim 10, wherein the linker further comprises a maleimide group.

12. The ADC of claim 10, wherein the linker further comprises a polyethylene glycol (PEG).

13. A composition comprising the ADC of claim 1 and a pharmaceutically acceptable carrier.

14. A method of treating a CD276-positive cancer in a subject, comprising administering a therapeutically effective amount of the ADC of claim 1 to the subject, thereby treating the CD276-positive cancer in the subject.

15. A method of inhibiting tumor growth or metastasis of a CD276-positive cancer in a subject, comprising administering a therapeutically effective amount of the ADC of claim 1 to the subject, thereby inhibiting tumor growth or metastasis of the CD276-positive cancer in the subject.

16. The method of claim 14, wherein the cancer is hepatocellular carcinoma, melanoma, leukemia, breast cancer, neuroblastoma, prostate cancer, colorectal cancer, osteosarcoma, endometrial cancer, ovarian cancer, oral squamous cell carcinoma, non-small cell lung cancer, bladder cancer or pancreatic cancer.

17. The method of claim 16, wherein the cancer is breast cancer or neuroblastoma.

18. The method of claim 14, further comprising administering to the subject an additional anti-cancer agent.

19. The method of claim 18, wherein the additional anti-cancer agent comprises a chemotherapeutic agent or an anti-angiogenesis agent.


SEQUENCE LISTING (7 sequences)

SEQ ID NO: 1 - protein, 19 aa, Artificial Sequence
  Synthetic peptide
MEWSWVFLFFLSVTTGVHS

SEQ ID NO: 2 - protein, 119 aa, Artificial Sequence
  Synthetic polypeptide
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSS

SEQ ID NO: 3 - protein, 330 aa, Artificial Sequence
  Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 449 aa, Artificial Sequence
  Synthetic polypeptide
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 468 aa, Artificial Sequence
  Synthetic polypeptide
MEWSWVFLFFLSVTTGVHSQVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 109 aa, Artificial Sequence
  Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIK

SEQ ID NO: 7 - protein, 216 aa, Artificial Sequence
  Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC